(19) United States
(12) Patent Application Publication
(10) Pub. No.: US 2005/0251007 A1
(43) Pub. Date: Nov. 10, 2005

(54) Device for use during medical procedures

(76) Inventor: Gary M. Ansel, Columbus, OH (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/830,949

(22) Filed: Apr. 22, 2004

(51) Int. Cl. A61B 5/05

(52) U.S. Cl. 600/407

(57) ABSTRACT

An instrument table for placement on an operating table on which a patient rests during a medical procedure includes a planar portion having a top surface and a bottom surface, the planar portion having a width less than the width of the operating table, at least one support coupled to the bottom surface of the planar portion for supporting the instrument table on the operating table, and a radiation shield, coupled to at least one of the planar portion and the at least one support.

### FIELD OF THE INVENTION

Embodiments of the present invention are directed to medical instrumentation, and more particularly to an instrument table and radiation protection for use during medical procedures.

### BACKGROUND OF THE INVENTION

Minimally invasive endovascular procedures have been developed to treat vascular diseases, such as blockage of the arteries. Some vascular blockages can be corrected by employing radiologic treatments that alleviate the need for invasive surgeries. Patients undergoing radiologic endovascular procedures are often awake, and therefore free to move around during the procedure. This creates concerns for the provider of the procedure. For example, the provider must carefully place his/her instruments in a convenient and easily accessible location while avoiding placement of the instruments in a location where the patient may disturb the instruments or be injured by the instruments.

During a radiologic procedure, the provider is also concerned with prolonged exposure to radiation, which can be highly harmful. For example, a provider may perform more than one radiology procedure each day, causing him/her to be exposed for a duration that is harmful. Also, procedures performed on the heart, hips and/or thighs typically take longer to complete, resulting in an increase of x-ray scatter, and therefore increased exposure. Typically, providers wear lead aprons for protection and not infrequently, providers also clip a meter to themselves that can alert them to overexposure conditions.

### SUMMARY OF THE INVENTION

It is desirable to provide a convenient and safe location for instrumentation needed during a medical procedure, particularly for procedures in which the patient may move around. It is further desirable to provide a radiation shield for medical providers to use during procedures requiring radiologic treatment.

The invention provides apparatus and methods for protecting against a provider's prolonged exposure to radiation during a medical procedure, while also providing a surface that increases efficient progression of a medical procedure without agitating the patient. An instrument table including side mounted radiation exposure shields is placed over the lower legs of a patient undergoing an upper body procedure, such as a cardiac or radiologic endovascular procedure. The instrument table provides a level surface onto which instruments are placed so that the instruments are conveniently accessible to the physician. Side mounted radiation pads provide protection from x-ray scatter. Throughout the procedure, the physician can be in close proximity to the patient undergoing radiologic treatment while not being overly concerned with overexposure from x-ray scatter.

In one embodiment, the invention provides an instrument table for placement on an operating table on which a patient rests during a medical procedure. The table provides a planar portion having a top surface and a bottom surface, the planar portion having a width less than the width of the operating table, at least one support coupled to the bottom surface of the planar portion for supporting the instrument table on the operating table, and a radiation shield, coupled to at least one of the planar portion and the at least one support.

The invention may include one or more of the following features. The instrument table includes a planar portion that is extendable in length. The planar portion includes an extendable portion on a topside of the instrument table that pulls out to an extended length over the patient. Alternatively, the extendable portion is sandwiched between two planar layers and is pulled out to extend the length of the instrument table. The height of the instrument table is also extendable or retractable by extending or retracting the at least one support. The at least one support is constructed and arranged to allow clearance beneath the planar portion for human legs to rest without contacting an underside of the planar portion, or with minimal contact with the underside of the planar portion of the table. The at least one support extends from a center of the planar portion such that a leg of the patient rests on each side of the at least one support. The at least one support may alternatively comprise a first side support and a second side support. The first side support and the second side support extend substantially perpendicularly from the planar portion to rest on the operating table straddling a portion of the legs of a patient resting on the operating table. Thus, in a first embodiment, the legs of the patient straddle the single table support, and in a second embodiment, the first side support and the second side support straddle the legs, collectively, of the patient.

The radiation shield comprises a first radiation pad coupled to a first side of the planar portion and a second radiation pad coupled to a second side of the planar portion. The first radiation pad and the second radiation pad are removably attached to the planar portion. The radiation shield extends below a surface of the operating table.

Accordingly, in one aspect, the invention features an instrument table having a horizontal surface, a right side support, and a left side support. The supports are positioned a distance from one another such that the supports straddle the legs of a patient lying on an operating table in position for a procedure. In particular, the instrument table straddles the lower portion of the legs of a patient, such as below the knee, so that procedure can be performed on an upper leg and upper body portion. The width of the instrument table is wider than the width of two human legs lying side-by-side and less wide than the width of a standard operating table on which a patient lies, which is about two feet in width. The instrument table sits substantially horizontally on an operating table without becoming unsteady due to movements by the patient. Alternatively, the instrument table rests on or contacts a portion of the legs of the patient. The instrument table has a height slightly greater than the thickness of a human leg from a front side to a dorsal side. The height of the instrument table is no lower than the height of the hip of the provider and no higher than the elbow height of the provider. Such a height allows for convenient access to instrumentation on the instrument table. The instrument table is made of a rigid plastic or of a heavy fiberboard. The instrument table is alternatively constructed of metal, other plastics, and other known materials. The instrument table is made of a radiation shielding material, for example.

Radiation pads are attached on the right side and the left side of the instrument table. The radiation pads extend from the top horizontal plane of the instrument table and extend to the top horizontal plane of the surgery table. Alternatively, the radiation pads hang below the horizontal planar portion of the operating table. The radiation pads are removably attached to the instrument table using any of a number of known attaching devices, such as snaps, VELCRO, tape, and zippers. Radiation pads can be longer than the length of the instrument table such that the radiation pads extend to a position over the upper legs and/or torso of the patient.

The invention will be more fully understood after a review of the following figures, detailed description and claims.

### BRIEF DESCRIPTION OF THE FIGURES

For a better understanding of the present invention, reference is made to the figures which are incorporated herein by reference and in which:

FIG. 1A is a top view of an instrument table having side supports in one embodiment of the invention;

FIG. 1B is a side perspective view of the instrument table of FIG. 1A;

FIG. 1C is a side perspective view of the bottom side of the instrument table of FIG. 1A;

FIG. 1D is a side perspective view of an extended instrument table in one embodiment of the invention;

FIGS. 2A-2B are views of the instrument table of FIG. 1A shown in use over the legs of a patient;

FIG. 3A is a perspective view of the prior art use of an operating table;

FIG. 3B is a perspective view of the instrument table of FIG. 1A in use;

FIG. 4 is a perspective view of the instrument table of FIG. 1A in use; and

FIG. 5 is a schematic view of an instrument table laid out flat.

### DETAILED DESCRIPTION OF THE INVENTION

The invention provides an instrument table for use during medical procedures performed on the upper body, including radiologic endovascular procedures. The invention provides radiation protection for the provider of the medical procedure. In one aspect, the invention provides an instrument table having a support, the support being positioned such that the table rests steadily on an operating or surgery table, the instrument table further having radiation pads on side portions to decrease radiation exposure. Embodiments of the invention can be used without the radiation pads and for medical procedures other than radiologic endovascular procedures. Still further embodiments are within the scope of the invention.

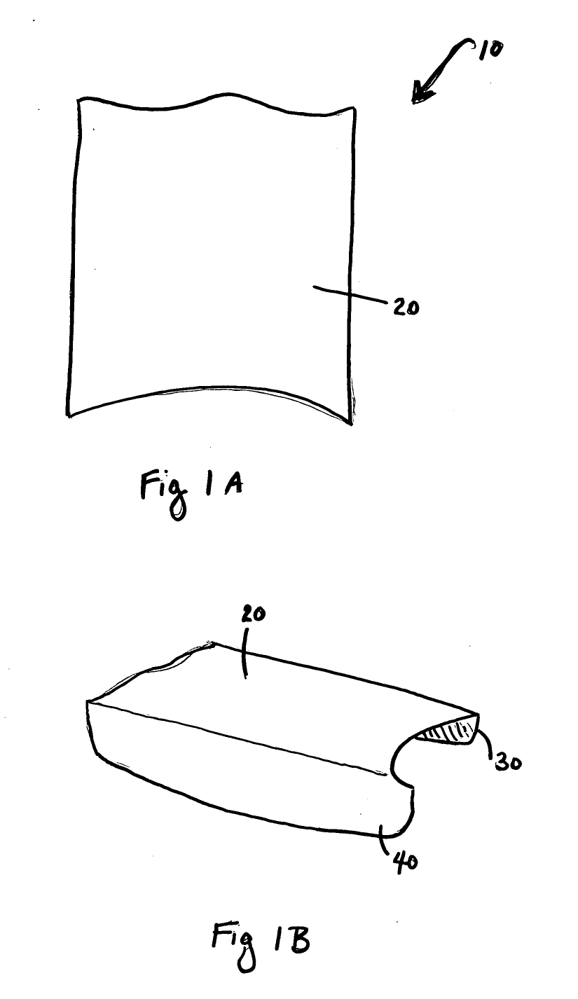

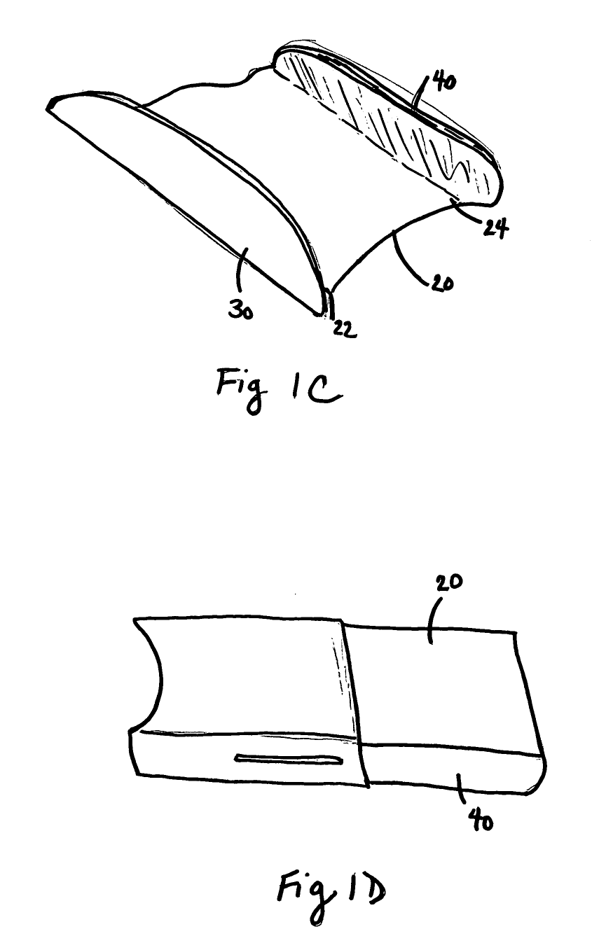

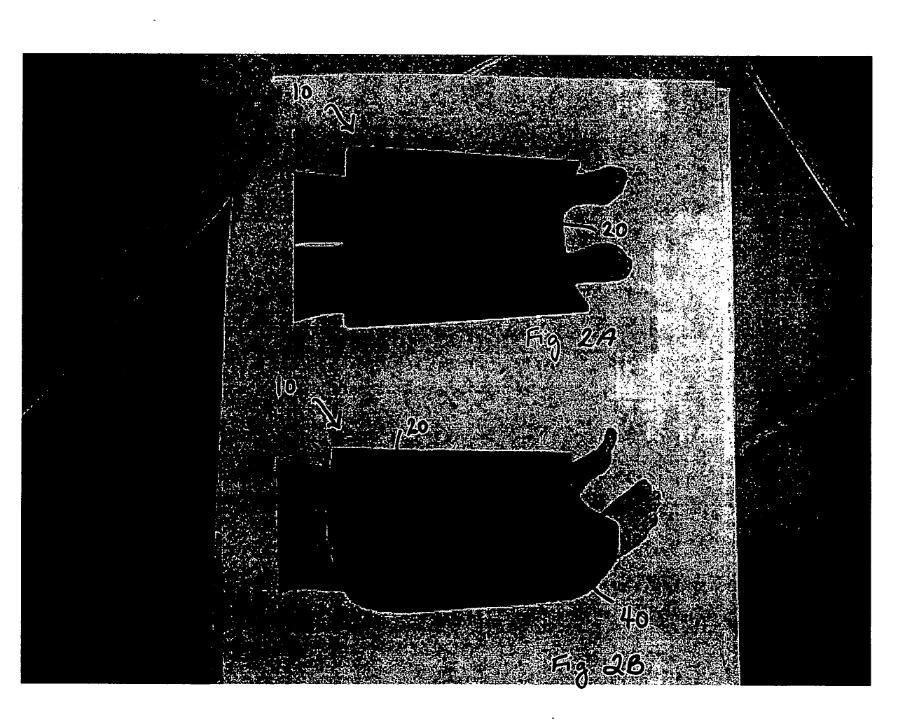

Referring to FIGS. 1A-1C, a medical instrumentation table, or a “lab slab” is shown. The lab slab 10 includes a top planar portion 20, a right side support 30, and a left side support 40. The right side support 30 extends substantially perpendicularly from a bottom right side 22 of the top portion. The left side support extends substantially perpendicularly from a bottom left side 24 of the top portion. The left and right side supports are the length of the top portion and are equivalent to one another in height. The lab slab is designed to rest on a planar surface, such as an operating table, for use during a medical procedure.

The right side support 30 and the left side support 40 are of the same height so that when resting on a substantially level surface, the lab slab is substantially horizontal on its top surface 20. The right side support and the left side support are 7 inches in height, for example. When placed on an operating table, the lab slab 10 creates a lengthwise aperture beneath the top planar portion and between the left and right side supports. The height of the side supports allows clearance beneath the top planar portion 20 for human legs to rest in the aperture without contacting the underside of the table 10, or with minimal contact with the underside of the table 10. The supports can be 6, 7, 8, 9, 10 or more inches in height to allow leg clearance. The supports are at a height such that when the lab slab is resting on an operating table, the top planar portion 20 of the lab slab rests at a level between the waist height and the elbow height of the physician or provider that is performing a medical procedure. At this height, the provider will not be uncomfortable when reaching for the instrumentation, i.e., the provider need not reach up above his/her mid-level for the tools.

The lab slab of FIGS. 1A-1C has a length covering from about the knee to about the ankle of the patient over which the lab slab is placed. The lab slab 10 may include an optional extension, shown in FIG. 1D. The extension 50 includes side portions 52 and a top portion 54 that is slightly larger than the underlying top portion 20. The extension side portions 52 are positioned at a width corresponding to the extension top portion 54 and slightly greater than the width of the underlying top portion 20. With such dimensions, the extension 50 is free to slide to an extended position that enlarges the available space on the top of the lab slab 10 and increase the coverage provided over the leg area of the patient on the operating table. The extension 50 may be useful for some procedures but not necessary in others.

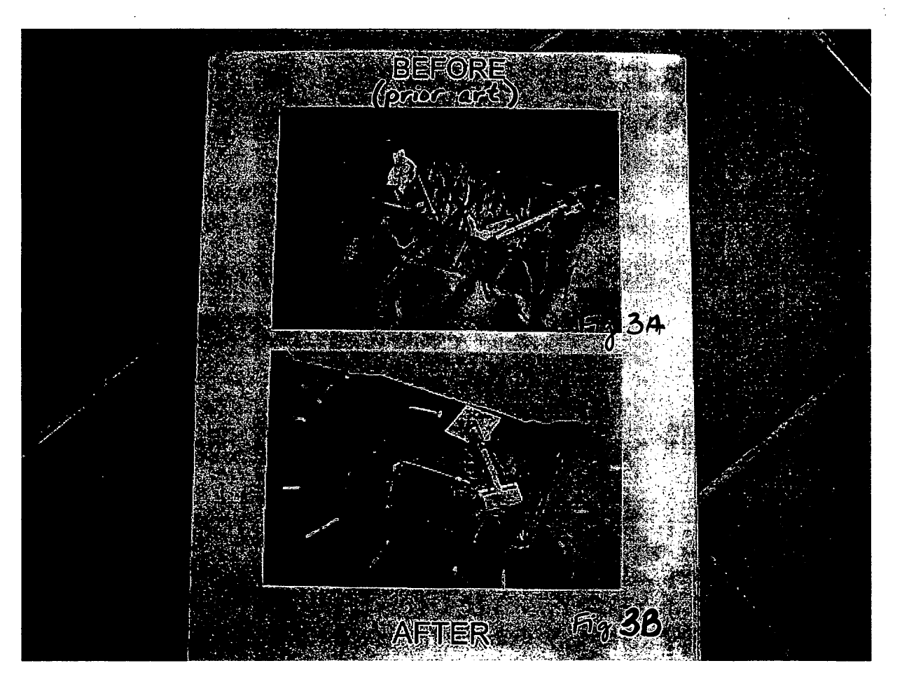

Referring to FIGS. 2A and 2B, the lab slab of FIGS. 1A-1C is shown in use over the lower legs of a patient. FIGS. 2A and 2B illustrate that the feet of the patient extend out of a bottom side of the lab slab. The patient's legs can rest easily side-by-side without discomfort. The height of the lab slab allows the patient to position his/her legs without substantially contacting the lab slab.

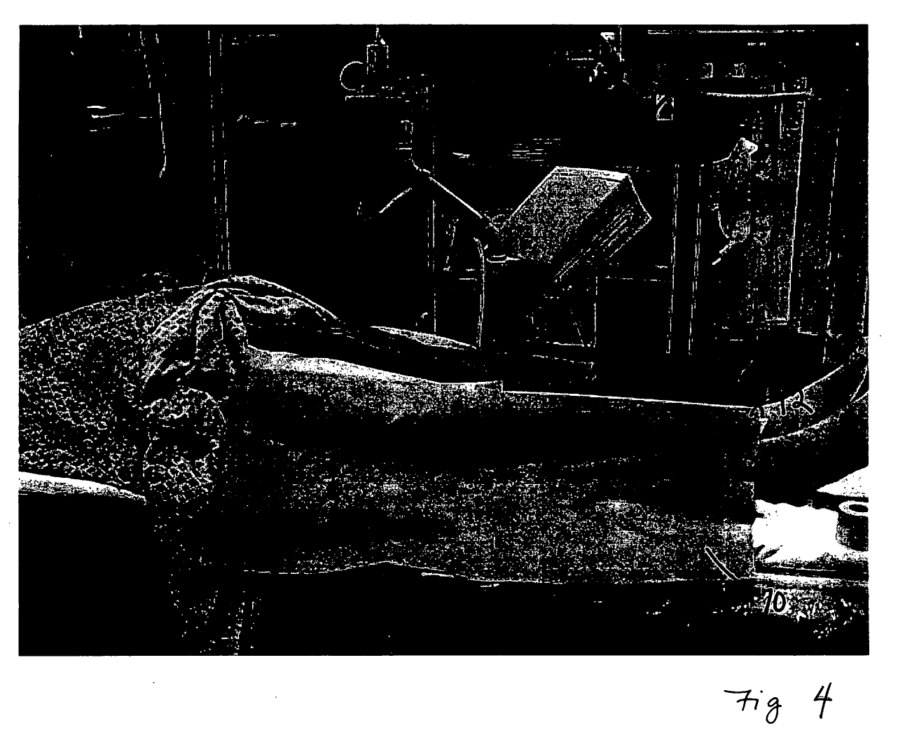

Using the lab slab during medical procedures increases the work area with which the provider has to work. Without the lab slab, instrumentation can become unorganized, inconveniently positioned, or harmful. Referring to FIG. 3A, instrumentation is shown placed on the legs of a patient who is resting on an operating table for a procedure, which is a common practice. In FIG. 3B, the lab slab 10 is shown in position over the legs of a patient. Instrumentation is placed on the top of the lab slab in preparation for use. As is shown, the lab slab of FIG. 3B increases the usable space available to the provider and medical assistants. The lab slab 10 further provides protection to the patient from inadvertent skin punctures from sharp instruments as well as doing away with the sensation of movement on or near the legs during a procedure in which the patient is awake.

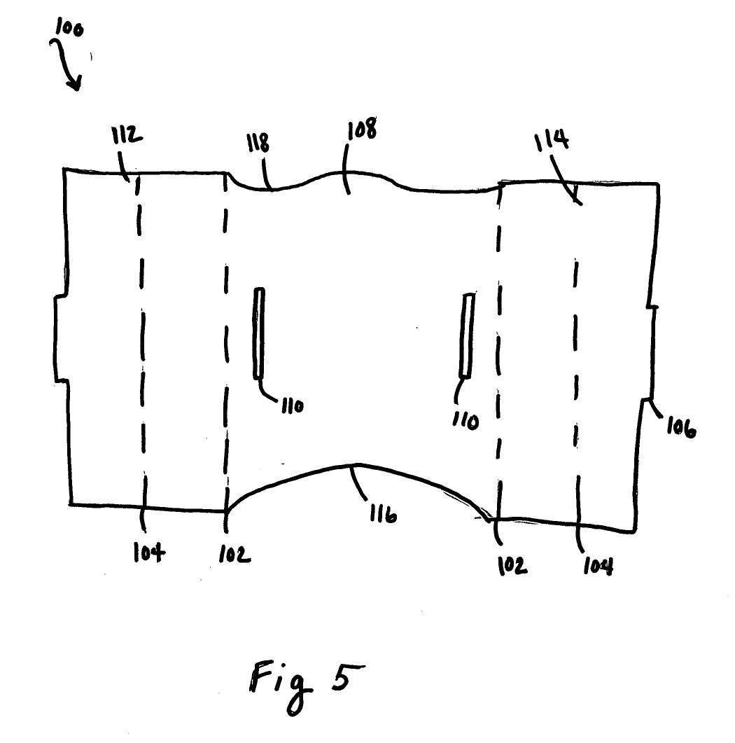

In FIG. 4, the lab slab is again shown in position over the lower legs of a patient lying on an operating table. Referring to FIG. 4, the lab slab 10 includes radiation pads 70 positioned on each side portion of the lab slab. The radiation pads 70 decrease exposure to x-ray scatter so that the provider does not become overexposed to radiation used during the procedure. The radiation pads can include adhesive at a topside portion 72. The adhesive accomplishes connection of the radiation pad with the edge of the lab slab 10. The adhesive used on the radiation pad can be any of a number of known adhesives, such as glue, tape, VELCRO, and others. For example, the radiation pad has the attributes of the radiation pad disclosed in U.S. Pat. No. 6,310,355, entitled, “Lightweight Radiation Shield System,” assigned to Worldwide Innovations and Technologies, Inc., of Overland Park, Kans. Other known radiation pads can be used for attachment to the table. Alternatively, the radiation pads 70 are coupled to the lab slab 10 during manufacture and are not removable.

As shown in FIG. 4, radiation pads 70 may extend past the edge of the lab slab 10 to cover a portion of the patient. Additional protection to the provider is thereby provided. The radiation pad 70 is a single pad having a length greater than the length of the lab slab. The length of the radiation pad may be the length of the legs of the patient. The radiation pad can fold out to a length twice as long as the length of the lab slab, or greater. The lab slab having radiation pads accomplishes the dual function described above.

As shown in FIGS. 1A-1C, the lab slab can be a single molded piece of plastic forming a horizontal surface having support members. The lab slab can be constructed of substantially rigid plastic, fiberboard, or any of a number of known materials, including metals. The lab slab can be disposable after a single use or reusable any of a number of times.

Exemplary dimensions of the lab slab are as follows. The top planar portion 20 is approximately 21 inches in width and 28 inches in length. The width of the top portion 20 is such that the top portion is slightly wider than the width of two human legs when positioned side-by-side, and slightly less wide than the width of a standard operating table, which is approximately 2 feet in width. The right side support and the left side support are 7 inches in height, measured from the top planar edge of the lab slab. The lab slab 10 is manufactured of a single piece of substantially rigid plastic, which is molded to include the substantially perpendicular right side and left side supports. The radiation pads are approximately 28 inches in length and have a width of at least 7 inches, but may be as wide as 8, 10, 12, or 14 inches to cover the side portion of the patient when on the operating table.

The lab slab of FIGS. 1A-1C is a single molded plastic body. With reference to FIG. 5, an alternative embodiment of the lab slab is shown, laid out flat. The lab slab can be assembled from a flat member, making it easy to store. In FIG. 5, the lab slab 100 includes a single body portion 108, side portions 112 and 114 having symmetrical folds 102 and 104, tabs 108, and slots 110. Side portions 112 and 114 have substantially identical dimensions. The side portions 112 and 114 are folded inward at folds 102 and 104. When folded inward, the tabs 108 on the side portions 112 and 114 fit into slots 110. The tabs 106 secure the side portions in a perpendicular position relative to the top portion. The side portions 112 and 114 thus fold into supports for the top portion 108, creating an instrumentation table. The top horizontal portion includes an arched bottom side 116 and a curved topside 118. The arched bottom 116 creates additional space for a patient's feet to move when the lab slab 100 is in position over the patient. The shape of the top portion 108 can, however, be any of a number of shapes, including square, oval, and rectangular.

Exemplary dimensions of the lab slab 100 shown in FIG. 5 are as follows. The lab slab has a length of 16 inches. The top portion can have a width of 20 inches. The distance from the internal fold lines (i.e., the fold lines closest to the top portion) to the external folds is 8 inches, and the distance from the external folds to the side edge of the lab slab is 8½ inches. Each tab can be 4 inches in length, and ½ inch wide. The tabs insert into slots that are 4 inches in length. The slots are positioned two inches from the internal folds. Again, dimensions are exemplary only and not intended to be limiting.

The embodiments of the invention described above include an instrumentation table having two supports. Other embodiments of the invention are acceptable, such as an instrument table having a single support positioned toward a mid portion of the top plane of the table. The support allows for the legs of a patient to straddle the support, such that the left leg is on one side of the support and the right leg is on an opposite side of the support. An additional acceptable embodiment includes a planar portion that rests on the legs of the patient during a medical procedure. In addition, acceptable embodiments of the invention include clips, slots, or apertures that hold wires and other equipment used during a medical procedure. Clips are provided at edges of the instrument table, for example, to keep the wire or wires from entanglement or from getting in the way of other instrumentation.

Having thus described at least one illustrative embodiment of the invention, various alterations, modifications and improvements will readily occur to those skilled in the art. Such alterations, modifications and improvements are intended to be within the scope and spirit of the invention. Accordingly, the foregoing description is by way of example only and is not intended as limiting. The invention's limit is defined only in the following claims and the equivalents thereto.

1. An instrument table for placement on an operating table on which a patient rests during a medical procedure, the instrument table comprising:
a planar portion having a top surface and a bottom surface, the planar portion having a width less than the width of the operating table;
at least one support coupled to the bottom surface of the planar portion for supporting the instrument table on the operating table; and
a radiation shield, coupled to at least one of the planar portion and the at least one support.

2. The instrument table of claim 1 wherein the planar portion is extendable in length.

3. The instrument table of claim 1 wherein the radiation shield comprises a first radiation pad coupled to a first side of the planar portion and a second radiation pad coupled to a second side of the planar portion.

4. The instrument table of claim 3 wherein the first radiation pad and the second radiation pad are removably attached to the planar portion.

5. The instrument table of claim 1 wherein the radiation shield extends below a surface of the operating table.

6. The instrument table of claim 1 wherein the at least one support is constructed and arranged to allow clearance beneath the planar portion for human legs to rest without contacting an underside of the planar portion.

7. The instrument table of claim I wherein the at least one support extends from a center of the planar portion such that a leg of the patient rests on each side of the at least one support.

8. The instrument table of claim 1 wherein the at least one support comprises a first side support and a second side support.

9. The instrument table of claim 8 wherein the first side support and the second side support extend substantially perpendicularly from the planar portion to rest on the operating table straddling a portion of the legs of a patient resting on the operating table.
